(19) United States
(12) Patent Application Publication
(10) Pub. No.: US 2026/0009791 A1
(43) Pub. Date: Jan. 8, 2026

(54) ANALYTICAL METHODS, DEVICES AND KITS FOR USE THEREIN

(71) Applicant: UNIVERSITY OF LINCOLN, Lincoln (GB)

(72) Inventors: Stefan Heber Millson, Lincoln (GB); Anna Wilkinson, Nettleham (GB)

(21) Appl. No.: 18/880,009

(22) PCT Filed: Jul. 3, 2023
(86) PCT No.: PCT/GB2023/051741

§ 371 Date: Dec. 30, 2024

(30) Foreign Application Priority Data
Jul. 4, 2022 (GB) 2209807.3

(51) Int. Cl. G01N 33/543; G01N 33/573

(52) U.S. Cl. CPC G01N 33/54388; C12Y 302/01001; G01N 33/573; G01N 2333/928; G01N 2800/7004

(57) ABSTRACT

The present invention provides a method for determining a state of negative arousal in a healthy animal from the order Carnivora to be tested, the method comprising: (i) providing one or more saliva sample(s) from said animal; (ii) measuring the concentration and/or amount of amylase in said sample; wherein a state of negative stress arousal is determined based on the concentration and/or amount of amylase measured. The invention also provides the use of salivary alpha amylase as a biomarker for determining a state of negative arousal and assay devices and kits for carrying out the methods of the invention.

The present invention relates to methods for determining a state of negative arousal in a healthy animal from the order Carnivora by measuring the concentration and/or amount of amylase in a saliva sample from said animal. The invention also provides assay devices and kits for carrying out said methods.

Certain environments can be challenging for domestic and captive animals and recognising signs of compromised welfare is difficult.

Current physiological measures of arousal or stress, though effective when used in conjunction with behavioural measures, have been criticised for being expensive whilst lacking stability and specificity. A major concern is their inability to differentiate between emotional states of contrasting valence (i.e. positive/negative)—a key requirement for welfare assessment. For example, increased cortisol/creatinine ratio might be observed when a dog experiences a new environment for the first time, but this does not reveal whether that experience was perceived as either positive or negative by the dog.

There is therefore an urgent requirement for a physiological measure that is not subject to these limitations; that is simple, quick and cheap to record; and allows differentiation between states of positive and negative valence.

Amylase expression is induced in mammals as part of the adrenal “fight or flight response” and is more stable to measure than adrenaline. Amylase is currently being tested in human clinical trials as the new “gold standard” to replace the cortisol assay for stress. It has been reported in a number of textbooks (e.g. Canine and Feline Nutrition, Linda P. Case et al, Third Edition, 2011, https://doi.org/10.1016/C2009-0-39175-8) that saliva of carnivores such as dogs does not contain the protein amylase. However, dog saliva does contain amylase but at a much lower level than in humans (Sanguansermsri et al., PLOS One, 13(12):e0208317, 2018). Humans are likely to express high levels of amylase in saliva due to a varied diet, which can contain a substantial amount of starch (amylase is the enzyme that degrades starch into simple sugars that our body can then use as energy).

The derived source of salivary amylase is from the blood plasma, and levels are normally measured in blood plasma. Amylase is a relatively small protein that passes through membranes with other small proteins that are carried across from the plasma to the saliva. Hong et al. (Journal of Veterinary Science, 20(5):e46, 2019) and Kang et al. (BMC Veterinary Research, 18:31) discuss the use of salivary alpha-amylase as a stress biomarker specifically in diseased dogs, and is focussed entirely on disease-induced stress. Contreas-Aguilar et al. (BMC Veterinary Research, 13:266, 2017) discusses the detection and measurement of alpha-amylase in canine saliva and changes in the specific context of an experimentally induced sympathetic activation through induction of ejaculation. However, there remains a need to develop an efficient means to determine states of negative arousal in normal healthy carnivores.

Herein, the inventors surprisingly discovered that salivary amylase levels correlate with negative arousal in healthy animals, as exemplified in dogs. Based on this, salivary amylase levels can be used to determine a state of negative arousal in healthy animals from the order Carnivora and can therefore be used as a diagnostic indicator. The benefits of measuring salivary amylase to characterise negative arousal in healthy animals include: potential for a low-cost method of detection of negative arousal; the ability to measure amylase levels using rapid test kits (i.e. no need to send the sample to a laboratory); the specificity of amylase based tests to negative arousal, meaning additional measures are not required to differentiate between negative and positive arousal; the ease and non-invasive nature of using saliva samples compared to blood samples.

A first aspect of the invention provides a method for determining a state of negative arousal in a healthy animal from the order Carnivora to be tested, the method comprising:
(i) providing one or more saliva sample(s) from said animal;
(ii) measuring the concentration and/or amount of amylase in said sample;
wherein a state of negative arousal is determined based on the concentration and/or amount of amylase measured.

The method of the invention can therefore be used to determine whether an animal is in a state of negative arousal.

By “arousal” we refer to the animal's state of response to various stimuli in the environment. These stimuli can be physical, environmental, or psychological, for example. A heightened state of arousal (either caused by positive or negative triggers) can lead to increased heart rate, increased blood pressure, increased sensory alertness, increased mobility, dilated pupils and excitability.

Arousal can either be positive or negative. This can be referred to as the “valence” of the arousal. “Positive arousal” refers to the state of arousal caused by activities or environments that cause pleasure. For example, in dogs, this may be caused by exciting events such as playing. “Negative arousal” refers to the state of arousal caused by activities or environments that cause negative stress or other negative emotions. For example, in dogs, this may be caused by being left alone or being in an unfamiliar environment (e.g. kennels). In some embodiments, negative arousal is also referred to as negative stress.

The methods of the invention can also be used to differentiate positive arousal from negative arousal. In some embodiments, the method can be used to determine a state of negative arousal in an animal that is known to be aroused (but the valence of arousal is unknown). The ability to differentiate between negative arousal and positive arousal is particularly useful as the action taken to reduce arousal differs depending on whether the arousal is positive or negative, which is often not clear. Additionally, the traditional cortisol test for determining arousal cannot differentiate between positive arousal and negative arousal. The method of the present invention is therefore surprisingly particularly useful for differentiating the type of arousal in healthy animals.

The method of the invention is for determining a state of negative arousal in a healthy animal. By “healthy animal” we mean one that is free from disease, and we also mean that the animal is not actively undergoing or knowingly will imminently undergo any treatment for a disease, including surgery to treat a disease. By “disease” we mean a state that differs from the normal functional state of the animal. Disease is generally caused by infectious agents, hereditary causes, deficiencies or physiological causes, and does not include obesity unless that itself causes a disease. In some embodiments, this means that the animal is free from disease that has been diagnosed by a veterinarian. A skilled person in this field will know how to determine if an animal is free from disease.

The method of the invention represents the first time that salivary amylase has been used to determine a state of negative stress in healthy animals. In other words, the state of negative arousal is not simply stress induced by disease. In some embodiments, the state of negative arousal is not induced by disease.

The animal subject to be tested in the methods of the present invention is from the order Carnivora.

The skilled person will be aware that organisms are classified according to seven taxonomic ranks, that are arranged in the following hierarchical order (from broadest to narrowest): kingdom; phylum; class; order; family; genus; and species. A single organism can therefore be classified according to each of these ranks. Organisms falling within the same rank will have a genetic relationship to each other.

In the context of the present invention, organisms falling within the order Carnivora are also referred to as Carnivorans. These organisms are placental mammals that are adapted to primarily eat flesh. Common names of organisms falling within the order Carnivora include: cats; hyenas; mongooses; civets; dogs; bears; raccoons; weasels; and seals.

In some preferred embodiments, the animal subject to be tested in the methods of the present invention is from the family Canidae or Felidae.

In some embodiments, the animal subject to be tested in the methods of the present invention is from the family Canidae. In some embodiments, the animal subject to be tested in the methods of the present invention is from the family Felidae.

In the context of the present invention, organisms falling within the family Canidae are also referred to as canids or dogs. Canids fall within the order Carnivora. Common names of organisms falling within the family Canidae include: domestic dogs; wolves; coyotes; foxes; jackals; and dingoes. Canids also include any other dog-like mammal.

In some embodiments, the animal to be tested in the method of the present invention falls within the genus Canis. Canis fall within the family Canidae and the order Carnivora. Common names of organisms falling within the genus Canis include: domestic dogs; wolves; coyotes; and jackals.

In some preferred embodiments, the animal to be tested in the context of the present invention is a dog. In some preferred embodiments, the animal to be tested in the context of the present invention is a domestic dog. In some embodiments, the domestic dog is referred to as its species name Canis familiaris or Canis lupus familiaris.

In some embodiments, the animal to be tested in the context of the present invention is a wolf.

Organisms falling within the family Felidae are referred to as felines or cats. Felids fall within the order Carnivora. Common names of organisms falling within the family Felidae include: domestic cats; tigers; lions; jaguars; leopards; bobcats; caracals; cheetahs; cougars; and ocelots. Felids also include any other cat-like mammal.

In some embodiments, the animal to be tested in the method of the present invention falls within the genus Felis. Felis fall within the family Felidae and the order Carnivora. Common names of organisms falling within the genus Felis include: domestic cats; European wildcats; African wildcats; Chinese mountain cats; sand cats; black-footed cats; and jungle cats.

In some preferred embodiments, the animal to be tested in the context of the present invention is a cat. In some preferred embodiments, the animal to be tested in the context of the present invention is a domestic cat. In some embodiments, the domestic cat is referred to as its species name Felis catus.

In some embodiments, the method of the present invention is useful for determining a state of negative arousal in captive wild animals from the order Carnivora, or the family Canidae or Felidae. For example, these may be captive wolves, tigers, lions, leopards etc. in zoos or wildlife parks. The method of the invention is particularly applicable to these animals as it is more difficult to determine whether these animals are in a state of negative arousal purely based on behavioural characteristics.

As discussed above, the method of the invention measures amylase in one or more saliva sample(s). By “amylase” we mean an enzyme that catalyses the hydrolysis of starch into sugars. By “salivary amylase” we refer to an amylase enzyme found within saliva. Typically, animals of the order Carnivora (to which Canidae and Felidae also belong) have low levels of amylase in their saliva when they are in a non-aroused state as these animals have low levels of dietary starch. For example, Cornell University College of Veterinary Medicine EClinPath (https://eclinpath.com/chemistry/pancreas/amylase/) states that the organ specificity of amylase production is as follows:
Pancreas: Found in zymogen granules. The pancreas has higher concentrations of amylase than other tissues.
Intestine: Duodenum, ileum
Ovary and testes
Salivary gland: Salivary amylase is found in high concentration in pigs, resulting in high reference intervals for amylase in this species. Dogs lack salivary amylase.

This means that salivary amylase has been found to be elevated in animals of the order Carnivora only in a state of arousal, and as determined herein, this elevation is much higher in a state of negative arousal compared to positive arousal. These animals express amylase from the pancreas, which is then secreted into the saliva under conditions of negative arousal.

For example, it is known that there is minimal digestive enzyme (i.e. amylase) activity in saliva of carnivores such as cats and dogs. The primary function of saliva in such animals is lubrication of food and protection of the oral mucosa. Saliva also has antimicrobial properties and buffering agents (see, for example, Understanding the anatomy of canine and feline salivary glands, Veterinary Practice News, 18 Sep. 2018). Therefore, the methods of the present invention are expected to be applicable to all such animals normally having minimal amylase in their saliva.

Salivary amylase includes “salivary alpha amylase” as discussed herein. Alpha amylase (also referred to as α-amylase) hydrolyses the alpha bonds of alpha linked polysaccharides.

Therefore, in some embodiments, the methods, uses, devices and kits of the present invention measure the concentration and/or amount of salivary alpha amylase.

The method of the present invention relies on the provision of one or more saliva samples for testing. These samples are also referred to herein as “test samples”. In some embodiments, the method comprises providing one or more saliva samples obtained from the subject to be tested, i.e. obtained before the methods of the invention are performed

By “saliva sample” we mean any sample of fluid taken or originating from the mouth or throat area of the animal to be tested, excluding blood. Saliva is an extracellular fluid secreted by the salivary glands. The typical makeup of saliva differs between species. However, the major components are: water; electrolytes; mucus; white blood cells; antimicrobial agents; and enzymes (e.g. amylase).

Saliva samples are typically obtained using a swab of the mouth area, for example by swabbing the inside of the cheeks or from a toy/ball/object the animal has taken in the mouth and the deposited saliva collected from the toy/ball/object using a swab. Other methods for obtaining saliva samples are well known in the art.

The methods of the present invention rely on measuring the concentration and/or amount of salivary amylase in the one or more saliva samples. By “concentration” we mean the amount of salivary amylase in a defined amount of saliva. For example, a concentration measurement in the context of the present invention is typically expressed as ng of amylase per ml of saliva (ng/ml). Concentration measurements can also be expressed as, for example: ng/ml, pg/μl, ng/μl, ng/nl, pg/nl etc. Methods of measuring specific protein concentration are discussed further below.

By “amount” we mean the absolute amount of salivary amylase in the sample, i.e. the measurement is independent of the sample volume. Measurements of amount can be expressed as mg, μg, ng, pg, or fg etc.

In some preferred embodiments, the methods of the present invention involve measurement of the concentration of salivary amylase in the one or more samples. In some preferred embodiments this is expressed in terms of ng of salivary amylase per ml of saliva.

In one embodiment, the methods of the present invention do not include measuring the activity of the amylase enzyme. Measurement of activity levels of salivary amylase do not necessarily accurately reflect the amount of the enzyme in the sample, when comparing between samples. This is because genetic variation in amylase genes means that not all animals will have comparable levels of amylase activity. Activity levels of amylase in saliva can also be influenced by dietary and environmental factors. It is therefore advantageous in some embodiments to measure concentration and/or amount of amylase rather than measuring activity levels, and this is especially important when comparing the concentration and/or amount of amylase to cut-off values to make a determination of a state of negative stress.

In some embodiments, the methods of the present invention involve comparing the concentration and/or amount of salivary amylase in the test sample(s) to the concentration and/or amount of salivary amylase in one or more control samples in order to determine whether the test subject is negatively aroused.

In some embodiments, the method of the present invention further comprises the steps of:
(iii) providing one or more control saliva sample(s) from an animal that is in a known state of negative arousal (i.e. a positive control),
(iv) measuring the concentration and/or amount of amylase in said control sample(s),
wherein the animal to be tested is determined to be in a state of negative arousal or the level of negative arousal is determined based on the concentration and/or amount of amylase in the control sample corresponding to the concentration and/or amount of amylase in the sample obtained from the animal to be tested.

By “corresponding to the concentration and/or amount of amylase in the sample obtained from the animal to be tested” we include that the concentration and/or amount is identical to that of a positive control sample; or closer to that of one or more positive control sample than to one or more negative control sample (or to predefined reference values representing the same). Preferably the presence and/or amount is within ±40% of that of the one or more control sample (or mean of the control samples), for example, within ±39%, ±38%, ±37%, ±36%, ±35%, ±34%, ±33%, ±32%, ±31%, ±30%, ±29%, ±28%, ±27%, ±26%, ±25%, ±24%, ±23%, ±22%, ±21%, ±20%, ±19%, ±18%, ±17%, ±16%, ±15%, ±14%, ±13%, ±12%, ±11%, ±10%, ±9%, ±8%, ±7%, ±6%, ±5%, ±4%, ±3%, ±2%, ±1%, ±0.05% or within 0% of the one or more control sample (e.g., the positive control sample).

Alternatively or additionally, the difference in the presence or amount in the test sample is ≤2 standard deviation from the mean presence or amount in the control samples, for example, ≤2, ≤1.5, ≤1.4, ≤1.3, ≤1.2, ≤1.1, ≤1, ≤0.9, ≤0.8, ≤0.7, ≤0.6, ≤0.5, ≤0.4, ≤0.3, ≤0.2, ≤0.1 or 0 standard deviations from the from the mean presence or amount in the control samples.

Alternatively or additionally, by “corresponds to the presence and/or amount in a control sample” we include that the presence or amount in the test sample correlates with the amount in the control sample in a statistically significant manner. By “correlates with the amount in the control sample in a statistically significant manner” we mean or include that the presence or amount in the test sample correlates with the that of the control sample with a p-value of ≤0.05, for example, ≤0.04, ≤0.03, ≤0.02, ≤0.01, ≤0.005, ≤0.004, ≤0.003, ≤0.002, ≤0.001, ≤0.0005 or ≤0.0001.

In some additional or alternative embodiments, the method further comprises the steps of:
(v) providing one or more control saliva sample(s) from an animal that is in a known non-aroused state and/or in a state of positive arousal (i.e. a negative control),
(vi) measuring the concentration and/or amount of amylase in said control sample(s),
   wherein the animal to be tested is determined to be in a state of negative arousal or the level of negative arousal is determined based on the concentration and/or amount of amylase in the control sample being different from the concentration and/or amount of amylase in the sample obtained from the animal to be tested. In some embodiments, by “being different from the concentration and/or amount of amylase in the sample obtained from the animal to be tested”, we mean that the concentration and/or amount of amylase in the negative control sample is lower than the concentration and/or amount of amylase in the test sample in order for a state of negative arousal to be determined.

By “being different from the concentration and/or amount of amylase in the sample obtained from the animal to be tested” we include that the concentration and/or amount of salivary amylase in the test sample differs from that of the one or more control sample(s) (or to predefined reference values representing the same). Preferably, the concentration and/or amount in the test sample differs from the presence or amount in one or more control sample(s) (or mean of the control samples) by at least ±5%, for example, at least ±6%, ±7%, ±8%, ±9%, ±10%, ±11%, ±12%, ±13%, ±14%, ±15%, ±16%, ±17%, ±18%, ±19%, ±20%, ±21%, ±22%, ±23%, ±24%, ±25%, ±26%, ±27%, ±28%, ±29%, ±30%, ±31%, ±32%, ±33%, ±34%, ±35%, ±36%, ±37%, ±38%, ±39%, ±40%, ±41%, ±42%, ±43%, ±44%, ±45%, ±41%, ±42%, ±43%, ±44%, ±55%, ±60%, ±65%, ±66%, ±67%, ±68%, ±69%, ±70%, ±71%, ±72%, ±73%, ±74%, ±75%, ±76%, ±77%, ±78%, ±79%, ±80%, ±81%, ±82%, ±83%, ±84%, ±85%, ±86%, ±87%, ±88%, ±89%, ±90%, ±91%, ±92%, ±93%, ±94%, ±95%, ±96%, ±97%, ±98%, ±99%, ±100%, ±125%, ±150%, ±175%, ±200%, ±225%, ±250%, ±275%, ±300%, ±350%, ±400%, ±500% or at least ±1000% of the one or more control sample(s) (e.g., the negative control sample).

Alternatively or additionally, the presence or amount in the test sample differs from the mean presence or amount in the control samples by at least >1 standard deviation from the mean presence or amount in the control samples, for example, ≥1.5, ≥2, ≥3, ≥4, ≥5, ≥6, ≥7, ≥8, ≥9, ≥10, ≥11, ≥12, ≥13, ≥14 or ≥15 standard deviations from the mean presence or amount in the control samples. Any suitable means may be used for determining standard deviation (e.g., direct, sum of square, Welford's), however, in one embodiment, standard deviation is determined using the direct method (i.e., the square root of [the sum the squares of the samples minus the mean, divided by the number of samples]).

Alternatively or additionally, by “is different to the presence and/or amount in a control sample” we include that the presence or amount in the test sample does not correlate with the amount in the control sample in a statistically significant manner. By “does not correlate with the amount in the control sample in a statistically significant manner” we mean or include that the presence or amount in the test sample correlates with that of the control sample with a p-value of >0.001, for example, >0.002, >0.003, >0.004, >0.005, >0.01, >0.02, >0.03, >0.04 >0.05, >0.06, >0.07, >0.08, >0.09 or >0.1. Any suitable means for determining p-value known to the skilled person can be used, including z-test, t-test, Student's t-test, f-test, Mann-Whitney U test, Wilcoxon signed-rank test and Pearson's chi-squared test.

In some preferred embodiments, a control sample is not required for comparison each time the method is carried out. As one example, instead, a pre-determined reference cut-off value can be used that can be applied each time the method is performed for a sample from a particular species.

By “cut-off value” we mean a value or range of values of salivary amylase concentration or amount, where if a test sample is measured to have a concentration or amount of salivary amylase above this value, a state of negative arousal is determined. Similarly, if a test sample is measured to have a concentration or amount of salivary amylase below the cut-off value, a state of negative arousal is not determined (i.e. the animal is said to not be negatively aroused).

In some embodiments, a state of negative arousal is determined if the concentration of amylase measured is greater than a value from about 3 ng/ml to 4 ng/ml, for example, greater than from about 3.1 ng/ml to 3.9 ng/ml, greater than from about 3.2 ng/ml to 3.8 ng/ml, greater than from about 3.3 ng/ml to 3.8 ng/ml, greater than from about 3.4 ng/ml to 3.8 ng/ml, greater than from about 3.5 ng/ml to 3.8 ng/ml, greater than from about 3.6 ng/ml to 3.8 ng/ml, greater than from about 3.7 ng/ml to 3.8 ng/ml, or greater than from about 3.75 ng/ml to 3.8 ng/ml.

In some additional or alternative embodiments, a state of negative arousal is determined if the concentration of amylase measured is about 3 ng/ml or greater, for example, about 3.1 ng/ml or greater, about 3.2 ng/ml or greater, about 3.3 ng/ml or greater, about 3.4 ng/ml or greater, about 3.5 ng/ml or greater, about 3.6 ng/ml or greater, about 3.7 ng/ml or greater, about 3.8 ng/ml or greater, about 3.9 ng/ml or greater, or about 4.0 ng/ml or greater.

In some additional or alternative embodiments, a state of negative arousal is determined if the concentration of amylase measured is about 3.7 ng/ml or greater, for example, about 3.71 ng/ml or greater, about 3.72 ng/ml or greater, about 3.73 ng/ml or greater, about 3.74 ng/ml or greater, about 3.75 ng/ml or greater, about 3.76 ng/ml or greater, about 3.77 ng/ml or greater, about 3.78 ng/ml or greater, or about 3.79 ng/ml or greater.

In some preferred embodiments, a state of negative arousal is determined in the concentration of amylase is about 3.76 ng/ml or greater.

In some preferred embodiments, when the test subject is a domestic dog a state of negative arousal is determined in the concentration of amylase is about 3.76 ng/ml or greater.

In some preferred embodiments, a state of negative arousal is determined in the concentration of amylase is about 4.00 ng/ml or greater.

In some preferred embodiments, when the test subject is a domestic dog a state of negative arousal is determined in the concentration of amylase is about 4.00 ng/ml or greater.

In some additional or alternative embodiments, a state of negative arousal is not determined if the concentration of amylase measured is from 0 ng/ml to about 4.0 ng/ml, for example from 0 ng/ml to about 3.9 ng/ml, from 0 ng/ml to about 3.8 ng/ml, from 0 ng/ml to about 3.7 ng/ml, from 0 ng/ml to about 3.6 ng/ml, from 0 ng/ml to about 3.5 ng/ml, from 0 ng/ml to about 3.4 ng/ml, from 0 ng/ml to about 3.3 ng/ml, from 0 ng/ml to about 3.2 ng/ml, from 0 ng/ml to about 3.1 ng/ml, from 0 ng/ml to about 3.0 ng/ml.

In some embodiments, a state of negative arousal is determined if the concentration of amylase measured is greater than a value from about 2.0 ng/ml to 3.0 ng/ml, for example, greater than from about 2.01 ng/ml to 2.99 ng/ml, greater than from about 2.01 ng/ml to 2.98 ng/ml, greater than from about 2.01 ng/ml to 2.97 ng/ml, greater than from about 2.02 ng/ml to 2.96 ng/ml, greater than from about 2.03 ng/ml to 2.95 ng/ml, greater than from about 2.04 ng/ml to 2.94 ng/ml, greater than from about 2.05 ng/ml to 2.93 ng/ml, greater than from about 2.06 ng/ml to 2.92 ng/ml, greater than from about 2.05 ng/ml to 2.91 ng/ml, greater than from about 2.04 ng/ml to 2.90 ng/ml, or greater than from about 2.05 ng/ml to 2.90 ng/ml.

In some additional or alternative embodiments, a state of negative arousal is determined if the concentration of amylase measured is about 2.0 ng/ml or greater, for example, about 2.01 ng/ml or greater, about 2.02 ng/ml or greater, about 2.03 ng/ml or greater, about 2.04 ng/ml or greater, about 2.05 ng/ml or greater, about 2.06 ng/ml or greater, about 2.07 ng/ml or greater, about 2.08 ng/ml or greater, about 2.09 ng/ml or greater, or about 2.1 ng/ml or greater.

In some preferred embodiments, a state of negative arousal is determined if the concentration of amylase is about 2.01 ng/ml or greater.

In some preferred embodiments, when the test subject is a domestic dog a state of negative arousal is determined if the concentration of amylase is about 2.01 ng/ml or greater.

In some preferred embodiments, a state of negative arousal is determined if the concentration of amylase is about 2.1 ng/ml or greater.

In some preferred embodiments, when the test subject is a domestic dog a state of negative arousal is determined if the concentration of amylase is about 2.1 ng/ml or greater.

In some preferred embodiments, a state of negative arousal is determined if the concentration of amylase is about 2.97 ng/ml or greater.

In some preferred embodiments, when the test subject is a domestic dog a state of negative arousal is determined if the concentration of amylase is about 2.97 ng/ml or greater.

In some preferred embodiments, a state of negative arousal is determined if the concentration of amylase is about 3.00 ng/ml or greater.

In some preferred embodiments, when the test subject is a domestic dog a state of negative arousal is determined if the concentration of amylase is about 3.00 ng/ml or greater.

In some additional or alternative embodiments, a state of negative arousal is not determined if the concentration of amylase measured is from 0 ng/ml to about 3.0 ng/ml, for example from 0 ng/ml to about 2.9 ng/ml, from 0 ng/ml to about 2.8 ng/ml, or from 0 ng/ml to about 2.7 ng/ml, or from 0 ng/ml to about 2.6 ng/ml, or from 0 ng/ml to about 2.5 ng/ml, or from 0 ng/ml to about 2.4 ng/ml, or from 0 ng/ml to about 2.3 ng/ml, or from 0 ng/ml to about 2.2 ng/ml, or from 0 ng/ml to about 2.1 ng/ml.

In some embodiments, a state of negative arousal is determined if the concentration of amylase measured is greater than a value from about 2 ng/ml to 3 ng/ml, for example, greater than from about 2.1 ng/ml to 2.9 ng/ml, greater than from about 2.2 ng/ml to 2.8 ng/ml, greater than from about 2.3 ng/ml to 2.7 ng/ml, or greater than from about 2.4 ng/ml to 2.6 ng/ml.

In some embodiments, a state of negative arousal is determined if the concentration of amylase measured is about 2 ng/ml or greater, for example, about 2.1 ng/ml or greater, about 2.2 ng/ml or greater, about 2.3 ng/ml or greater, about 2.4 ng/ml or greater, about 2.5 ng/ml or greater, about 2.6 ng/ml or greater, about 2.7 ng/ml or greater, about 2.8 ng/ml or greater, about 2.9 ng/ml or greater, or about 3.0 ng/ml or greater.

In some embodiments, a state of negative arousal is determined if the concentration of amylase measured is: (i) about 2 ng/ml or greater, for example, about 2.01 ng/ml or greater, about 2.02 ng/ml or greater, about 2.03 ng/ml or greater, about 2.04 ng/ml or greater, about 2.05 ng/ml or greater, about 2.06 ng/ml or greater, about 2.07 ng/ml or greater, about 2.08 ng/ml or greater, or about 2.09 ng/ml or greater; or (ii) 2.90 ng/ml or greater, for example, about 2.91 ng/ml or greater, about 2.92 ng/ml or greater, about 2.93 ng/ml or greater, about 2.94 ng/ml or greater, about 2.95 ng/ml or greater, about 2.96 ng/ml or greater, about 2.97 ng/ml or greater, about 2.98 ng/ml or greater, or about 2.99 ng/ml or greater.

In some embodiments, a state of negative arousal is determined if the concentration of amylase measured is greater than a value from about 1 ng/ml to 6 ng/ml, for example, from about 2 ng/ml to 5 ng/ml, or from about 3 ng/ml to 4 ng/ml.

The skilled person would be aware that the cut-off values for determining a state of negative arousal may differ, for example depending on the family, genus, or species of animal to be tested.

The methods of the present invention may utilise a single saliva sample in order to determine a state of negative arousal. In some embodiments, the methods may utilise more than one saliva sample to determine a state of negative arousal. For example, the methods may utilise two, three, four, five, six, seven, eight, nine, or ten or more separate saliva sample to determine a state of negative arousal. The multiple saliva samples may be taken at the same time point, and act as repeat measurements of the same time point. This may be useful to improve accuracy of the determination of negative arousal.

In additional or alternative embodiments, the multiple saliva samples may be taken at different time points. In this embodiment, the different saliva samples can provide information on the time course of salivary amylase levels in the animal, and the methods of the invention therefore provide information on the levels of negative arousal over time. This embodiment of the invention may be useful for determining whether a state of negative arousal persists over time (i.e. determining how long it lasts for), or whether negative arousal occurs following a particular trigger.

For example, saliva samples may be taken at various time points before an event that is suspected to cause a state of negative arousal. Samples may be taken, for example, up to 48 hours before the event, for example up to 24 hours before, up to 12 hours before, up to 10 hours before, up to 8 hours before, up to 6 hours before, up to 5 hours before, up to 4 hours before, up to 3 hours before, up to 2 hours before, up to 1 hour before, or up to 30 minutes before the event.

Similarly, samples may be taken at various time points after an event that is suspected to cause a state of negative arousal. Samples may be taken, for example, up to 48 hours after the event, for example up to 24 hours after, up to 12 hours after, up to 10 hours after, up to 8 hours after, up to 6 hours after, up to 5 hours after, up to 4 hours after, up to 3 hours after, up to 2 hours after, up to 1 hour after, or up to 30 minutes after the event.

In some other embodiments, samples may be taken at least 30 minutes after an event that is suspected to cause a state of negative arousal, for example at least 45 minutes after, at least 1 hour after, at least 2 hours after, at least 6 hours after, at least 12 hours after, or at least 24 hours after. In some other embodiments, samples are taken between 30 minutes and 1 hour after the event that is suspected to cause a state of negative arousal, or between 1 hour and 2 hours after the event, or between 2 hours and 6 hours after the event, or between 6 hours and 12 hours after the event, or between 12 hours and 24 hours after the event.

In some preferred embodiments, a sample is taken at a time point between 20 and 40 minutes after the event that is suspected to cause a state of negative arousal. In some preferred embodiments, a sample is taken at around 20 minutes after the event that is suspected to cause a state of negative arousal. In some other preferred embodiments, a sample is taken at around 30 minutes after the event that is suspected to cause a state of negative arousal. In some other preferred embodiments, a sample is taken at around 40 minutes after the event that is suspected to cause a state of negative arousal. In some alternative embodiments, a sample is taken at around 1 hour after the event that is suspected to cause a state of negative arousal.

In taking samples at various time points before and after the event that is suspected to cause a state of negative arousal, it can be determined when the state of negative arousal occurs based on when the increase in salivary amylase is observed. Typically, it would be expected that salivary amylase would begin to increase around 15 to 30 minutes after the start of the event that causes a state of negative arousal. The salivary amylase levels would be expected to peak around 20 to 40 minutes after the start of the event that causes a state of negative arousal. Salivary amylase levels would be expected to begin to decrease only after the end of the event that causes negative arousal has ended, and they would decrease to normal levels by around 2 hours after the end of the event that causes negative arousal.

In some embodiments, the concentration and/or amount of salivary amylase in the test sample is proportional to the level of negative arousal determined. In this way, the methods of the present invention can be used to determine the level of negative arousal in the test subject.

In some embodiments, the concentration and/or amount of salivary amylase in the test sample correlates with the concentration of cortisol in saliva.

In some embodiments, the concentration and/or amount of salivary amylase is measured using a first binding agent capable of binding salivary amylase.

By “capable of binding” we mean that the binding agent binds the target (in this case, salivary amylase) more specifically than it binds to other proteins. This term may be used interchangeably with “specifically binds”.

In some embodiments, the first binding agent is an antibody or an antigen binding fragment thereof. In some embodiments, the antibody or antigen binding fragment thereof is a recombinant antibody or antigen binding fragment thereof. In some embodiments, the antibody or antigen binding fragment thereof is a monoclonal antibody or antigen binding fragment thereof. In some embodiments, the antibody or antigen binding fragment thereof is selected from the group consisting of: scFv; Fab; or a binding domain of an immunoglobulin molecule.

In some embodiments, the binding agent is an antibody or an antigen-binding fragment thereof, or a variant thereof.

Methods for the production and use of antibodies are well known in the art, for example see Antibodies: A Laboratory Manual, 1988, Harlow & Lane, Cold Spring Harbor Press, ISBN-13: 978-0879693145, Using Antibodies: A Laboratory Manual, 1998, Harlow & Lane, Cold Spring Harbor Press, ISBN-13: 978-0879695446 and Making and Using Antibodies: A Practical Handbook, 2006, Howard & Kaser, CRC Press, ISBN-13:978-0849335280 (the disclosures of which are incorporated herein by reference).

Thus, a fragment may contain one or more of the variable heavy (V_{H}) or variable light (V_{L}) domains. For example, the term antibody fragment includes Fab-like molecules (Better et al (1988) Science 240, 1041); Fv molecules (Skerra et al (1988) Science 240, 1038); single-chain Fv (scFv) molecules where the V_{H} and V_{L} partner domains are linked via a flexible oligopeptide (Bird et al (1988) Science 242, 423; Huston et al (1988) Proc. Natl. Acad. Sci. USA 85, 5879) and single domain antibodies (dAbs) comprising isolated V domains (Ward et al (1989) Nature 341, 544).

The term “antibody variant” includes any synthetic antibodies, recombinant antibodies or antibody hybrids, such as but not limited to, a single-chain antibody molecule produced by phage-display of immunoglobulin light and/or heavy chain variable and/or constant regions, or other immunointeractive molecule capable of binding to an antigen in an immunoassay format that is known to those skilled in the art.

A general review of the techniques involved in the synthesis of antibody fragments which retain their specific binding sites is to be found in Winter & Milstein (1991) Nature 349, 293-299.

Molecular libraries such as antibody libraries (Clackson et al, 1991, Nature 352, 624-628; Marks et al, 1991, J Mol Biol 222(3): 581-97), peptide libraries (Smith, 1985, Science 228(4705): 1315-7), expressed cDNA libraries (Santi et al (2000) J Mol Biol 296(2): 497-508), libraries on other scaffolds than the antibody framework such as affibodies (Gunneriusson et al, 1999, Appl Environ Microbiol 65(9): 4134-40) or libraries based on aptamers (Kenan et al, 1999, Methods Mol Biol 118, 217-31) may be used as a source from which binding molecules that are specific for a given motif are selected for use in the methods of the invention.

In some preferred embodiments, the binding agent is a whole antibody. In some preferred embodiments, the binding agent is a monoclonal antibody.

In some embodiments, the concentration and/or amount of salivary amylase is measured using an assay comprising a second binding agent capable of binding to salivary amylase, the second binding agent having a detectable moiety.

In some embodiments, the second binding agent is an antibody or an antigen binding fragment thereof. In some embodiments, the antibody or antigen binding fragment thereof is a recombinant antibody or antigen binding fragment thereof. In some embodiments, the antibody or antigen binding fragment thereof is selected from the group consisting of scFv, Fab and a binding domain of an immunoglobulin molecule.

For example, the first binding agent may initially be used to ‘trap’ the salivary amylase on to the surface of an array, and then a second binding agent may be used to detect the ‘trapped’ salivary amylase.

As discussed above, the second binding agent has a detectable moiety. By a “detectable moiety” we include the meaning that the moiety is one which may be detected and the relative amount determined.

Suitable detectable moieties are well known in the art. For example, the detectable moiety may be selected from the group consisting of: a fluorescent moiety; a luminescent moiety; a chemiluminescent moiety; a radioactive moiety; an enzymatic moiety.

In one preferred embodiment, the detectable moiety is biotin.

Thus, the detectable moiety may be a fluorescent and/or luminescent and/or chemiluminescent moiety which, when exposed to specific conditions, may be detected. For example, a fluorescent moiety may need to be exposed to radiation (i.e., light) at a specific wavelength and intensity to cause excitation of the fluorescent moiety, thereby enabling it to emit detectable fluorescence at a specific wavelength that may be detected.

Alternatively, the detectable moiety may be an enzyme which is capable of converting a (preferably undetectable) substrate into a detectable product that can be visualised and/or detected. Examples of suitable enzymes are discussed in more detail below in relation to, for example, ELISA assays.

In a further alternative, the detectable moiety may be a radioactive atom which is useful in imaging. Suitable radioactive atoms include ^{99m}Tc and ¹²³I for scintigraphic studies. Other readily detectable moieties include, for example, spin labels for magnetic resonance imaging (MRI) such as ¹²³I again, ¹³¹I, ¹¹¹In, ¹⁹F, ¹³C, ¹⁵N, ¹⁷O, gadolinium, manganese or iron. Clearly, the agent to be detected (such as, for example, the one or more biomarkers in the test sample and/or control sample described herein and/or an antibody molecule for use in detecting a selected protein) must have sufficient of the appropriate atomic isotopes in order for the detectable moiety to be readily detectable.

The skilled person will be aware of common techniques for the measurement of the concentration and/or amount of salivary amylase in a sample. Any technique for measuring the concentration and/or amount of an enzyme in a sample would be suitable for carrying out the methods of the present invention. Such techniques include, but are not limited to: Enzyme-Linked Immunosorbent Assay (ELISA); and Surface Plasmon Resonance (SPR) based techniques. Alternatively, the amylase in the sample could be specifically purified (e.g. using affinity chromatography or HPLC) and the total protein in the sample quantified using common techniques (e.g. Nanodrop or similar).

In some preferred embodiments, the concentration and/or amount of salivary amylase is determined by ELISA. ELISA methods are well known in the art, for example see The ELISA Guidebook (Methods in Molecular Biology), 2000, Crowther, Humana Press, ISBN-13: 978-0896037281 (the disclosures of which are incorporated by reference).

ELISA typically involves the use of enzymes giving a coloured reaction product, usually in solid phase assays. Enzymes such as horseradish peroxidase and phosphatase have been widely employed. A way of amplifying the phosphatase reaction is to use NADP as a substrate to generate NAD which now acts as a coenzyme for a second enzyme system. Pyrophosphatase from Escherichia coli provides a good conjugate because the enzyme is not present in tissues, is stable and gives a good reaction colour. Chemi-luminescent systems based on enzymes such as luciferase can also be used.

In some embodiments, the methods of the present invention are for diagnosis of a state of negative arousal in the animal.

In some embodiments, the methods of the present invention further comprise the step of providing the animal with treatment to address the negative arousal condition.

The treatment can be anything that eliminates the state of negative arousal in the animal. Alternatively, the treatment can be anything that reduces the severity of negative arousal in the animal. As a further alternative, the treatment can be anything that prevents further occurrences of negative arousal.

For example, these treatments may include: removing the cause of the negative arousal from the environment; or administering a medication to mitigate the effects of negative arousal or prevent it from occurring (e.g. a sedative). In some embodiments, the treatment includes administering a sedative medication. Common sedative medications for use in animals that may be used in accordance with the present invention are: acepromazine; diphenhydramine; gabapentin; trazodone; or benzodiazepines. The skilled person in this field will be aware of other common treatments to reduce or prevent a state of negative arousal.

The methods of the present invention find particular utility in their application to a rapid test device to determine a state of negative arousal. For example, this can be done by using a lateral flow device. This allows a sample to be obtained and applied directly to a test cartridge after mixing with a running buffer. A result can be obtained within around 30 minutes from the sample being taken. This allows a result to be obtained very quickly, and the appropriate treatment or intervention provided rapidly.

The discussion of each of the embodiments discussed above in relation to the first aspect of the invention applies equally to the second to ninth aspects of the invention.

A second aspect of the invention relates to use of salivary amylase as a biomarker for determining whether a healthy animal from the order Carnivora is in a state of negative arousal.

A third aspect of the invention relates to use of salivary amylase as a biomarker for determining whether a state of arousal in a healthy animal from the order Carnivora is positive arousal or negative arousal.

A fourth aspect of the invention relates to use of salivary amylase as a biomarker for determining the level of negative arousal in a healthy animal from the order Carnivora.

In some embodiments of these aspects, the use comprises measuring the concentration and/or amount of salivary amylase.

In some embodiments of these aspects, the healthy animal is from the family Canidae or Felidae.

A fifth aspect of the invention relates to use of a binding agent for salivary amylase for determining whether a healthy animal from the order Carnivora is in a state of negative arousal.

A sixth aspect of the invention relates to use of a binding agent for salivary amylase for determining whether a state of stress in a healthy animal from the order Carnivora is positive arousal or negative arousal.

A seventh aspect of the invention relates to use of a binding agent for salivary amylase for determining the level of negative arousal in a healthy animal from the order Carnivora.

An eighth aspect of the invention relates to an assay device for determining a state of negative arousal in a healthy animal from the order Carnivora, the device comprising:
(a) a sample receiving region for receiving a saliva sample taken from a healthy animal from the order Carnivora; and
(b) a capture region comprising an immobilised binding agent that binds specifically to salivary amylase of an animal from the order Carnivora.

In some embodiments, the assay device is a lateral flow assay device.

A skilled person will be aware of the basic components of a lateral flow assay device. A lateral flow assay device according to the present invention comprises at least:
(a) a sample receiving region for receiving a saliva sample taken from a healthy animal from the order Carnivora;
(b) a capture region comprising a first immobilised binding agent that binds specifically to salivary amylase of an animal from the order Carnivora.

Typically, the sample is mixed with a running buffer solution prior to application onto the sample receiving region. The running buffer allows the sample material to be carried laterally along the capture region. The running buffer can be any suitable buffer, for example compositions comprising phosphate buffered saline (PBS). The components of the running buffer may be adjusted to change the flow speed. The capture region is typically a membrane material, for example a nitrocellulose membrane. The capture region and sample receiving region may be housed in a container.

The running buffer also comprises a first labelled binding agent that is capable of binding to the salivary amylase. The salivary amylase binds to the labelled binding agent comprised in the running buffer. When the running buffer-sample mixture is applied to the sample receiving region and moves through the capture region, the salivary amylase binds to the first immobilised binding agent. This allows the labelled binding moieties to become immobilised to the capture region in the region where the first immobilised binding agents are found, and the labels can then be observed visually. The labels used typically include gold nanoparticles, carbon nanoparticles, and fluorescent nanoparticles, for example. The skilled person will know which labels are appropriate to use in lateral flow assay devices.

In some embodiments, the running buffer further comprises a binding partner and the lateral flow assay device also comprises a second immobilised binding agent in the capture region that binds specifically to the binding partner contained in the running buffer solution. The second immobilised binding agent is immobilised on a different portion of the capture region to the first immobilised binding agent. The interaction of the binding partner in the running buffer with the immobilised second binding agent acts as a control, showing that the running buffer has successfully migrated laterally along the capture region. In these embodiments, the running buffer further comprises a second labelled binding agent that is capable of binding to the binding partner. The binding partner binds to second labelled binding agent comprised in the running buffer. When this is applied to the sample receiving region and moves through the capture region, the binding partner binds to the second immobilised binding agent. This allows the second labelled binding agent to become immobilised to the capture region, and the labels can then be observed visually.

A lateral flow assay device can be used to visually determine whether an analyte (in this case salivary amylase) is present in a sample. Lateral flow assay devices can also be used to infer the concentration and/or amount of salivary amylase in a sample, based on obtaining a positive result in an assay configured to do so about a certain pre-determined cut-off value. For instance, as described herein, in some embodiments an animal is determined to be in a state of negative arousal if the salivary amylase is about 3.76 ng/ml or greater, in which case a lateral flow assay device can be configured to provide a positive result when salivary amylase is present at about 3.76 ng/ml or greater. The intensity of the positive result then reflects the concentration or amount of salivary amylase in the sample above this level.

A ninth aspect of the invention relates to a kit comprising:
(a) a binding agent that specifically binds salivary amylase of an animal from the order Carnivora;
(b) instructions for use.

In some embodiments, the kit further comprises one or more of the following:
(c) an assay device comprising one or more of the components described herein;
(d) running buffer for use with the lateral flow assay device described herein;
(e) materials for collecting one or more saliva samples from an animal (e.g. one or more swabs);
(f) containers for preparing the saliva samples for testing (e.g. one or more reservoir tubes).

In some embodiments, the assay device is a lateral flow assay device. In some embodiments of any of the above aspects, the binding agent is an antibody or an antigen binding fragment thereof. In some embodiments the binding agent specifically binds salivary amylase of an animal from the order Carnivora. In some embodiments the binding agent specifically binds salivary amylase of an animal from the family Canidae or Felidae. In some embodiments the binding agent specifically binds salivary amylase of a domestic dog or a domestic cat.

A further aspect of the invention provides the methods, uses, devices and kits substantially as described herein with reference to the description and drawings.

Preferred, non-limiting examples which embody certain aspects of the invention will now be described, with reference to the following figures and examples:

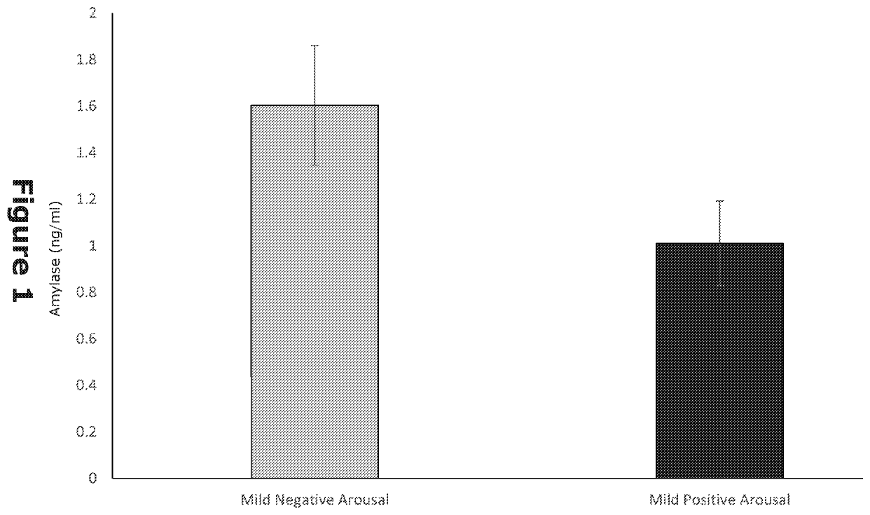

FIG. 1: Comparison of salivary amylase levels between the mild negative arousal and mild positive arousal conditions. Error bars are standard error. This shows that the mild negative arousal conditions produced a significantly higher amylase concentration in the saliva (mean 1.60 ng/ml) than the mild positive arousal condition (mean 0.96 ng/ml; t (11)=2.18, p≤0.05).

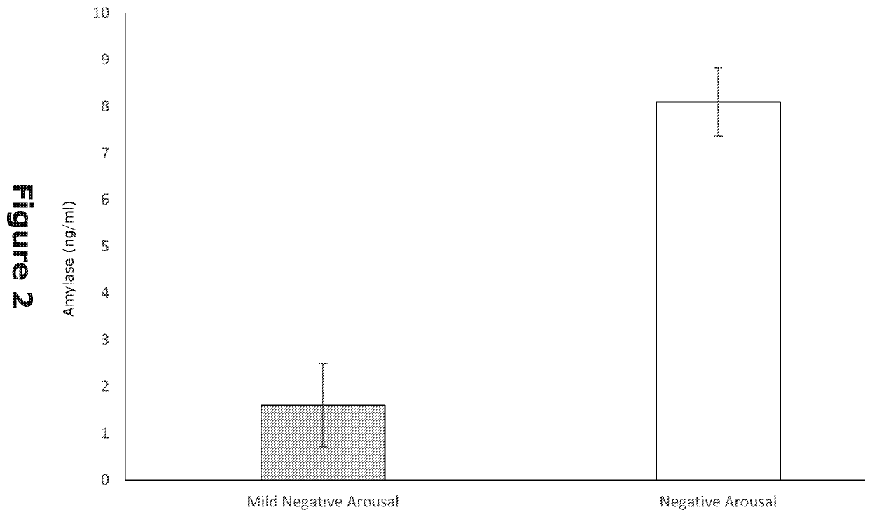

FIG. 2: Comparison of salivary amylase levels between mild negative arousal condition and the negative arousal condition. Error bars are standard error. This indicates that the negative arousal condition results in a 5-fold increase in salivary amylase concentration compared to the mild negative arousal condition

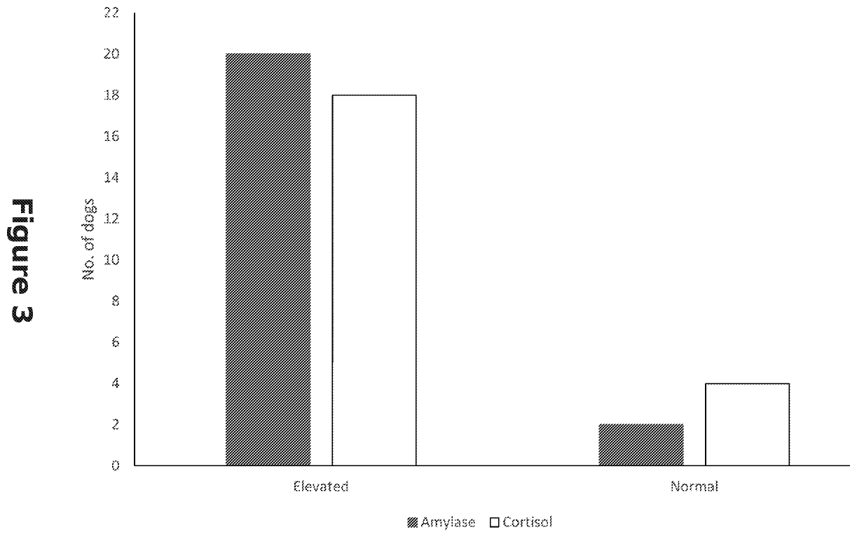

FIG. 3: Comparison of salivary amylase and cortisol levels in dogs with elevated (outside reference range) and within normal reference range. This indicates that there is no significant difference between the salivary cortisol and amylase tests in their ability to identify a dog as having elevated levels or levels within normal reference range.

### EXAMPLES

The inventors examined salivary amylase in response to positive and negative arousal situations in dogs.

### Example 1

### Methods

Subjects: Adult dogs were recruited through the Lincoln dog database (www.lincolnpetscando.co.uk). Inclusion criteria were as follows: dogs over 1 year of age; comfortable with separation from owners; engages in toy play; is motivated by toys; and no major health concerns.

Materials: The experiment took place in Minster house at Lincoln University, Lincolnshire, U.K. In both test situations the dogs were presented with their favourite toy that the owners bring with them. The toy was placed in a clear plastic box, which was anchored to a wooden board. Closing and securing the lid of the box created an unsolvable task (Marshall-Pescini et al., 2013; Miklósi et al., 2003) for the mild negative arousal test. To create a comparable positive test where the dog could access the toy, the lid was placed on top of the container but could easily be pushed off by the dog. Multiple cameras were positioned for video recording of behaviour. A mat was provided in a quite area for the dog to rest following the task, prior to second salivary sampling.

Procedure: A within-subject design was used to compare changes in salivary amylase levels before and after eliciting mild positive and mild negative arousal. The owner was not present during the testing.

Habituation phase: The dog was brought into the testing room and allowed to investigate the room off lead. Habituation lasted for a minimum of 3 minutes or until the dog sat or lay down and relaxed. During this time, the researcher remained seated and did not interact with the dog.

Saliva sampling: Collection of salivary samples occurred at two time points using nylon brush FLOQswabs™ (Copan Ltd). The first sample was collected after habituation, prior to testing, and the second sample was taken 30 minutes after testing. The dog was shown the saliva swab and allowed to sniff and familiarise itself with it prior to sampling. During sample collection the researcher crouched next to the dog and gently held on to its muzzle, the swab was inserted into the inside the dog's cheeks then then slowly rotated three times for a maximum total of 30 seconds. Samples were then stored on ice until frozen at −80° C. for storage prior to analysis.

Mild positive arousal test: To induce mild positive arousal, the toy was used to elicit play. The researcher presented the toy to the dog then placed it in a clear plastic box, in the middle of the room. The lid was balanced on top of the container so that it could easily be pushed off by the dog. The test began when the researcher moved away from the box said the dog's name once and pointed to and looked at the box. The dog could then approach the box to retrieve its toy. If the dog stopped trying to retrieve the toy after 15 seconds, then this action was repeated. If the dog had not successfully retrieved the toy after 30 seconds, the researcher removed the toy from the box and gave it to the dog. Once the dog had the toy the researcher encouraged it to play with the toy for 3 minutes. This trial time as used as it has been shown that cortisol levels change with this amount of play (Horváth et al., 2008). Play included tug-of-war and throwing the toy 1-2 metres away to be retrieved. It did include any commands such as sit, drop or stay. After 3 minutes of play the researcher stopped actively engaging in the play bout. If the dog approached the researcher they were offered their hand to sniff. Once the dog stopped interacting with the toy (maximum of 2 minutes) then the researcher removed it from the room before returning to sit on the chair. The dog was allowed to rest in a calm environment for the next 30 minutes.

Mild negative arousal test: To induce mild negative arousal the dog was prevented from accessing a desired object (toy). This condition was identical to the positive arousal test except when the toy was placed in the clear plastic box the lid was securely fastening so that the dog could not access the toy. The behaviour of the researcher remained the same except that the dog was not given the toy. After 3 minutes the box was removed from the room by the researcher before returning to sit on the chair.

Negative arousal test: A battery of tests were developed to assess frustration in dogs (for methodological details see McPeake et al., 2021). These included a questionnaire for owners and behavioural indicators. As part of this work, saliva samples were taken to assess cortisol levels. For the current study, the samples were also analysed to assess amylase levels.

Saliva analysis: Samples were defrosted at room temperature. The swab was cut at 1 cm above the end flocculation of the swab bud. The Swab bud was then transferred in an inverted position to a 1.5 ml low protein binding locking 1.5 ml Eppendorf tube (Eppendorf Ltd), centrifuged at 6,000×g for 1 minute and the swab removed. Alpha-Amylase levels were measured using an ELISA assay (Antibodies-online.com) and Cortisol was measured using an ELISA assay (Arbor Assays Ltd), following manufacturer's instructions. Saliva samples were diluted 1:5 prior to analysis, with the ELISA sample buffer supplied.

### Results & Discussion

Mild negative and positive arousal: The results of the analysis of amylase levels in the mild negative and positive arousal conditions revealed that the mild negative arousal condition produced a significantly higher amylase concentration in the saliva (mean 1.60 ng/ml) than the mild positive arousal condition (mean 0.96 ng/ml; t (11)=2.18, p≤0.05). This is shown in FIG. 1.

This finding reveals that amylase levels can be used to detect even mild negative arousal. Further, the change in arousal is specific to the mild negative arousal condition (but not positive arousal). This shows that, unlike cortisol, amylase is predominantly a marker for negative arousal in dogs.

Comparison of mild negative arousal condition with negative arousal condition: The results revealed that dogs in the negative arousal condition had a significantly higher salivary amylase concentration (mean 8.10 ng/ml) than the dogs in the mild negative arousal condition (mean 1.60 ng/ml; t(32)=6.41, p<0.0001).

The data in FIG. 2 indicates that the negative arousal condition results in a 5-fold increase in salivary amylase concentration compared to the mild negative arousal condition. This shows that as well as assessing the presence and absence of negative arousal, amylase may also be useful for assessing the level of negative arousal.

Correlation between amylase and behavioural markers in the negative arousal condition: The cohort of dogs from the negative arousal condition also had a behavioural measure of negative emotional state. There was a moderate positive correlation between amylase levels and the behavioural measure (total number of vocalisations) r=0.46, p≤0.05.

This finding reveals that amylase levels increase with behavioural markers of negative arousal (see, e.g. FIGS. 1 and 2).

Correlation between amylase and Canine Frustration Questionnaire data in the negative arousal condition: Owners of the cohort of dogs from the negative arousal condition also filled in the Canine Frustration Questionnaire (McPeake et al., 2019). A correlation between overall questionnaire score and amylase revealed a significant moderate effect r=0.48, p≤0.05.

This correlation between amylase levels and owner perception of frustration provides further evidence that amylase levels can be used as a good predictor of negative arousal in dogs.

Generation of normal reference range of salivary amylase in dogs: In the study described herein, the inventors analysed over 40 different dogs both male and female, common breeds and cross-breeds, plus ages from 1 year to 12 years in age, pre and post-arousal events. Dogs were tested at multiple timepoints and for most of the dogs, control measurements of cortisol levels were measured (current “gold standard” test for stress), as well as alpha amylase levels as discussed herein.

On the basis of this, the inventors calculated a preliminary reference range for dogs (Reference range=Mean+2× standard deviations). This was calculated from 12 dogs, each measured at three separate time points, on different days to allow for day-to-day variation.

This resulted in a mean saliva alpha amylase level of 1.41 ng/ml with a standard deviation of 0.78. This produces a “normal” reference range of 0 to 2.97 ng/ml.

Cortisol levels of 2 ng/ml is commonly regarded as the cut-off for moderate stress (Dreschel & Granger, 2009; Di Nardo et al., 2016). This is therefore be considered to be comparable to the maximum value of our reference range which is 2.97 ng/ml.

Given this data, the cut-off levels of saliva alpha amylase in dogs are:
0-2.9 ng/ml=Normal (i.e. no negative arousal);
3+ ng/ml (i.e. 3.0 ng/ml and greater)=Negative arousal

### Comparison Between Amylase and Cortisol Levels

Dogs were categorised as having either normal or elevated levels of both amylase and cortisol. A McNemar's test revealed no significant difference between cortisol and amylase measures X² (1, N=22)=0.25 p=0.62.

As shown in FIG. 3, there is no significant difference between the salivary cortisol and amylase tests in their ability to identify a dog as having elevated levels or levels within normal reference range. This further validates the usefulness of salivary amylase as a measure of negative arousal.

### Conclusions

Mild negative arousal conditions produced a significantly higher amylase concentration in the saliva than mild positive arousal suggesting that amylase is a marker for negative arousal, unlike cortisol which increases under both negative and positive stress.
Negative arousal conditions had a 5-fold higher amylase concentration in the saliva than mild negative arousal condition, showing that amylase is a useful measure of negative arousal levels.
Amylase levels correlated with behavioural measures of negative arousal.
Amylase levels correlated with owner questionnaire information on negative arousal in dogs.
When dogs in the negative arousal condition were categorised on the basis of cortisol and amylase as having normal or elevated levels of biomarker, there was no difference between the two measures.

These findings confirm that amylase is an excellent biomarker for negative arousal in dogs. This allows, for the first time, the rapid identification of negative arousal in dogs in real-time, for example by using rapid lateral flow testing to identify dogs with amylase levels outside the normal reference range.

### Example 2

The inventors repeated the study described in Example 1 for a cohort of 22 dogs. The mean amylase level for non-stressed dogs was found to be 1.25 ng/ml, with a standard deviation of 0.38 ng/ml for dogs under mild positive and mild negative arousal conditions.

The cohort was made-up of dogs of nine different breeds or mixed breeds, aged between 2 to 13 years, and each dog had baseline (non-stress) saliva samples taken in duplicate and on two separate days/occasions for the mild negative or positive arousal.

This compares closely to Example 1 which found the mean amylase level for 12 dogs of various breeds to be 1.41 ng/ml and a standard deviation of 0.78 ng/ml. The standard deviation of this cohort was higher due to the lower number of subjects included.

This data provides a “normal” reference range (Reference range=Mean+2× Standard deviations) of 0 to 2.01 ng/ml.

Given this data, the cut-off levels of saliva alpha amylase in dogs may alternatively be:
0-2 ng/ml=Normal (i.e. no negative arousal);
2.1+ ng/ml (i.e. 2.1 ng/ml and greater)=Negative arousal

Variation due to breed, sex, or age of the dogs outside normal deviation was not observed.

This evidences the reproducibility and robustness of measuring the amylase biomarker, which has stable low levels under normal mild positive and mild negative arousal.

### REFERENCES

Di Nardo, F., Anfossi, L., Ozella, L., Saccani, A., Giovańnoli, C., Spano, G., & Baggiani, C. (2016). Validation of a qualitative immunochromatographic test for the noninvasive assessment of stress in dogs. Journal of chromatography. B, Analytical technologies in the biomedical and life sciences, 1028, 192-198
Dreschel, N. A., & Granger, D. A. (2009). Methods of collection for salivary cortisol measurement in dogs. Hormones and behavior, 55(1), 163-168.
Horváth Z, Dóka A, Miklósi A. Affiliative and disciplinary behavior of human handlers during play with their dog affects cortisol concentrations in opposite directions. Horm Behav. 2008 June; 54(1):107-14.
Marshall-Pescini, S., Barnard, S., Branson, N. J., & Valsecchi, P. (2013). The effect of preferential paw usage on dogs' (Canis familiaris) performance in a manipulative problem-solving task. Behavioural processes, 100, 40-43.
McPeake, K.J., Collins, L.M., Zulch, H. and Mills, D.S., 2021. Behavioural and Physiological Correlates of the Canine Frustration Questionnaire. Animals, 11(12), p. 3346.
Miklósi, A., Kubinyi, E., Topál, J., Gácsi, M., Virányi, Z., & Csányi, V. (2003). A simple reason for a big difference: wolves do not look back at humans, but dogs do. Current biology: CB, 13(9), 763-766.

1. A method for determining a state of negative arousal in a healthy animal from the order Carnivora to be tested, the method comprising:
(i) providing one or more saliva sample(s) from said animal;
(il) measuring the concentration and/or amount of amylase in said sample;
wherein a state of negative arousal is determined based on the concentration and/or amount of amylase measured.

2. The method of claim 1, wherein the method is for determining a state of negative arousal in a healthy animal from the order Carnivora to be tested that is already known to be stressed.

3. The method of claim 1, wherein the method is for determining the level of negative arousal in a healthy animal from the order Carnivora to be tested.

4. The method of any of the preceding claims, wherein the animal to be tested is from the family Canidae or Felidae; and/or wherein the animal to be tested is from the genus Canis or Felis.

5. The method of any of the preceding claims, wherein the animal to be tested is a domestic dog or a domestic cat.

6. The method of claim 5, wherein the animal to be tested is a domestic dog.

7. The method of any of the preceding claims, wherein the amylase is salivary alpha amylase.

8. The method of any of the preceding claims, wherein a state of negative arousal is determined if the concentration of amylase measured is greater than a value from about 2 ng/ml to 3 ng/ml, for example, greater than from about 2.1 ng/ml to 2.9 ng/ml, greater than from about 2.2 ng/ml to 2.8 ng/ml, greater than from about 2.3 ng/ml to 2.7 ng/ml, or greater than from about 2.4 ng/ml to 2.6 ng/ml.

9. The method of claims 1-7, wherein a state of negative arousal is determined if the concentration of amylase measured is about 2 ng/ml or greater, for example, about 2.1 ng/ml or greater, about 2.2 ng/ml or greater, about 2.3 ng/ml or greater, about 2.4 ng/ml or greater, about 2.5 ng/ml or greater, about 2.6 ng/ml or greater, about 2.7 ng/ml or greater, about 2.8 ng/ml or greater, about 2.9 ng/ml or greater, or about 3.0 ng/ml or greater.

10. The method of claim 9, wherein a state of negative arousal is determined if the concentration of amylase measured is: (i) about 2 ng/ml or greater, for example, about 2.01 ng/ml or greater, about 2.02 ng/ml or greater, about 2.03 ng/ml or greater, about 2.04 ng/ml or greater, about 2.05 ng/ml or greater, about 2.06 ng/ml or greater, about 2.07 ng/ml or greater, about 2.08 ng/ml or greater, or about 2.09 ng/ml or greater; or (ii) 2.90 ng/ml or greater, for example, about 2.91 ng/ml or greater, about 2.92 ng/ml or greater, about 2.93 ng/ml or greater, about 2.94 ng/ml or greater, about 2.95 ng/ml or greater, about 2.96 ng/ml or greater, about 2.97 ng/ml or greater, about 2.98 ng/ml or greater, or about 2.99 ng/ml or greater.

11. The method of claim 10, wherein a state of negative arousal is determined if the concentration of amylase is about 2.01 ng/ml or greater or the concentration of amylase is about 2.97 ng/ml or greater.

12. The method of claims 1-7, wherein a state of negative arousal is not determined if the concentration of amylase measured is from 0 ng/ml to about 3.0 ng/ml, for example from 0 ng/ml to about 2.9 ng/ml, from 0 ng/ml to about 2.8 ng/ml, from 0 ng/ml to about 2.7 ng/ml, from 0 ng/ml to about 2.6 ng/ml, from 0 ng/ml to about 2.5 ng/ml, from 0 ng/ml to about 2.4 ng/ml, from 0 ng/ml to about 2.3 ng/ml, from 0 ng/ml to about 2.2 ng/ml, from 0 ng/ml to about 2.1 ng/ml, from 0 ng/ml to about 2.0 ng/ml.

13. The method of any of the preceding claims, further comprising the step of providing the animal with treatment to address the negative arousal, optionally wherein said treatment comprises one or more of the following: removing the cause of negative arousal from the environment, and administering a medication to mitigate the effects of the negative arousal.

14. The method of any of the preceding claims, wherein the method further comprises the steps of:
(iii) providing one or more control saliva sample(s) from an animal that is in a known state of negative arousal,
(iv) measuring the concentration and/or amount of amylase in said control sample(s),
wherein the animal to be tested is determined to be in a state of negative arousal or the level of negative arousal is determined based on the concentration and/or amount of amylase in the control sample corresponding to the concentration and/or amount of amylase in the sample obtained from the animal to be tested; and/or
wherein the method further comprises the steps of:
(v) providing one or more control saliva sample(s) from an animal that is in a known non-stressed state and/or in a state of positive stress,
(vi) measuring the concentration and/or amount of amylase in said control sample(s),
wherein the animal to be tested is determined to be in a state of negative arousal or the level of negative arousal is determined based on the concentration and/or amount of amylase in the control sample being different from the concentration and/or amount of amylase in the sample obtained from the animal to be tested.

15. The method of any of the preceding claims, wherein step (i) and/or step (iv) is performed using a first binding agent capable of binding to salivary amylase.

16. The method of claim 15, wherein the first binding agent is an antibody or an antigen binding fragment thereof; optionally wherein the antibody or antigen binding fragment thereof is a recombinant antibody or antigen binding fragment thereof; optionally wherein the antibody or antigen binding fragment thereof is selected from the group consisting of: scFv; Fab; a binding domain of an immunoglobulin molecule.

17. The method of claim 15 or 16, wherein step (i) and/or step (iv) is performed using an assay comprising a second binding agent capable of binding to salivary amylase, the second binding agent having a detectable moiety.

18. The method of claim 17, wherein the second binding agent is an antibody or an antigen binding fragment thereof; optionally wherein the antibody or antigen binding fragment thereof is a recombinant antibody or antigen binding fragment thereof; and/or wherein the antibody or antigen binding fragment thereof is selected from the group consisting of scFv, Fab and a binding domain of an immunoglobulin molecule.

19. The method of any of the preceding claims, wherein step (i) and/or step (iv) is performed using an enzyme-linked immunosorbent assay (ELISA).

20. The method of 1-18, wherein the method is performed using a lateral flow device.

21. Use of salivary amylase as a biomarker for determining whether a healthy animal from the order Carnivora is in a state of negative arousal; and/or determining whether a state of arousal in a healthy animal from the order Carnivora is positive arousal or negative arousal; and/or determining the level of negative arousal in a healthy animal from the order Carnivora.

22. Use of a binding agent for salivary amylase for determining whether a healthy animal from the order Carnivora is in a state of negative arousal; and/or determining whether a state of stress in a healthy animal from the order Carnivora is positive arousal or negative arousal; and/or determining the level of negative arousal in a healthy animal from the order Carnivora.

23. An assay device for determining a state of negative arousal in a healthy animal from the order Carnivora, the device comprising:
(a) a sample receiving region for receiving a saliva sample taken from a healthy animal from the order Carnivora; and
(b) a capture region comprising a first immobilised binding agent that binds specifically to salivary alpha amylase of an animal from the order Carnivora,
optionally wherein the assay device is a lateral flow assay device.

24. A kit comprising:
(a) a binding agent that specifically binds salivary alpha amylase of an animal from the order Carnivora;
(b) instructions for use.

25. The assay device or kit of claims 23-24, wherein the binding agent is an antibody or an antigen-binding fragment thereof, optionally wherein the antibody or antigen-binding fragment thereof is capable of binding to salivary amylase from an animal of the family Canidae or Felidae, optionally wherein the antibody or antigen-binding fragment thereof is capable of binding to salivary amylase from a domestic dog.
